(19) United States
(12) Patent Application Publication
(10) Pub. No.: US 2008/0279884 A1
(43) Pub. Date: Nov. 13, 2008

(54) Ingestible Plaque Eliminator for Animals

(76) Inventor: Richard Hellyer, East Dundee, IL (US)

Correspondence Address: Robert L. Knechtel;Law Offices of Mark E. Wiemelt, P.C., Suite 3300, 10 S. LaSalle Street, Chicago, IL, 60603, US

(21) Appl. No.: 11/747,895

(22) Filed: May 12, 2007

(51) Int. Cl. A61K 36/05; A61K 36/48; A61Q 11/00

(52) U.S. Cl. 424/195.17; 424/757

(57) ABSTRACT

An ingestible formulation for a plaque eliminator for animals that works systemically and comprises a formulation of a chlorophyll or a chlorophyll derivative combined with Spirulina and alfalfa wherein the chlorophyll derivative is at least 90 percent of the formulation is presented. In the preferred embodiment, the chlorophyll derivative is chlorophyllin. The formulation may be a capsule, liquid, tablet, or gelcap when in its usable form with the preferred embodiment being in a capsule formulation. When produced in tablet form, the formulation may also include a tableting agent. The formulation has a dissolution time generally sufficient to allow the formulation to pass an animal's stomach before beginning to breakdown. The formulation is dose dependent and will vary depending on the size of the animal to which it is given.

### I. TECHNICAL FIELD

The present invention relates to plaque reducing products for animals and, more particularly, to a plaque eliminator for animals which is ingested and provides plaque elimination properties through systemic mechanisms as opposed to localized effects.

### II. BACKGROUND OF THE INVENTION AND PRIOR ART

Plaque is constantly being made and deposited in an animal's mouth. It forms on an animal's teeth within 30 minutes of eating. It is a mixture of bacteria, minerals from saliva, and gummy food particles. Bacteria-filled Plaque persists if not brushed off daily. Soft at first, plaque gradually hardens and turns brown. Once hardened it is referred to as tartar. As more forms, it gets up under the gums, carrying infection. The bacteria cause bad breath, infection, and potentially more serious health problems.

In the absence of proper dental care, plaque can harden into tartar within 24 hours. Eventually there is soft tissue damage and bone loss around the tooth, which is referred to as periodontitis.

Periodontal disease, a general term for dental disease, is by far the number one most diagnosed problem in small animals today. By the age of two, 70% of cats and 80% of dogs have some degree of periodontal disease. As teeth become loose and infection spreads, the teeth become painful.

Periodontal disease not only causes tooth loss and bad breath, but also many local and systemic problems. Untreated periodontal disease is the single greatest cause of health problems in small animals. Research in humans shows that untreated periodontal disease is a key factor in the development of heart, liver and kidney problems and is a source of chronic low-grade infection. In fact, in women periodontal disease is a contributing factor to osteoporosis, birth defects, bone loss, and increased arthritis pain.

Preventing and treating periodontal disease can significantly add to the quality of an animal's life. However. It is not always possible to recognize signs of dental pain in an animal.

Over the years, many attempts to address the problem of dental plaque in animals have been advanced. Attempts have generally involved the use of chew toys or edible products that have a reducing agent contained within them, teeth brushing, or a mouth wash. Many formulations for product ingredients have been used, including, among others, chlorhexidine, alcohol and hydrogen peroxide based products. More recently, products containing chlorophyll or a derivative thereof have been used.

However, the general shortcoming of the prior art, including those containing chlorophyll or a derivative, is that they are focused on the oral cavity and do not provide a systemic solution. Accordingly, prior art products, if they have any true efficacy at all, provide immediate, temporary relief.

Another short coming of the prior art is that they generally use ingredients which are not generally optimally effective for the reason that they generally not fresh.

Another short coming of the prior art is that they generally contains an ineffective amount of active ingredient.

Yet another shortcoming of the prior art is that they are of a “one size fits all” variety; they do not take in to account the weight of the animal being given the deodorizer.

There is need, therefore, for an ingestible plaque eliminator for animals which provides systemic effects on a longer term basis, and is provided in clinically effective amounts based upon animal weight. The present invention provides such a solution.

### III. OBJECTS AND ADVANTAGES OF THE PRESENT INVENTION

It is an object of the present invention to provide a plaque eliminator for animals that contains effective levels of chlorophyll or a derivative.

It is a further object of the present invention to provide a plaque eliminator wherein the chlorophyll derivative is chlorophyllin.

It is a further object of the present invention to provide a plaque eliminator that contains alfalfa that functions as, among other things, as a balancing agent.

It is a further object of the present invention to provide a plaque eliminator that further contains Spirulina that functions as, among other things, a nutrient source.

It is a further object of the present invention to provide a plaque eliminator for animals that is ingestible.

It is yet a further object of the present invention to provide a plaque eliminator for animals that is available in a capsule, liquid, tablet, gelcap, or other ingestible form.

It is yet a further object of the present invention to provide a plaque eliminator for animals that is formulated to weight specifications of animals.

It is yet a further object of the present invention to provide a plaque eliminator for animals that works systemically.

It is yet a further object of the present invention to provide a plaque eliminator for animals that works on a longer term basis.

It is yet a further object of the present invention to provide a plaque eliminator for animals that has a dissolution time sufficient that the formulation passes an animal's stomach before beginning to breakdown.

Those skilled in the arts will see additional objects and advantages of the present invention, which invention is not to be limited by the foregoing objects and advantages paragraphs. Those skilled in the arts will also recognize additional formulations, methods, and systems for carrying out the several purposes of the present invention. It is important, therefore, that the claims be regarded as including such equivalent constructions in so far as they do not depart from the spirit and scope of the present invention. These together with other objects of the present invention, along with the various features of novelty which characterize the present invention, are pointed out with particularity in the claims annexed to and forming a part of this disclosure.

Further, the purpose of the abstract is to enable the US Patent and Trademark Office and the public generally, and especially the scientists, engineers and practitioners in the art who are not familiar with the patent or legal terms or phraseology, to determine quickly from what cursory inspection the nature and essence of the technical disclosure of the application. The abstract is neither intended to define the invention of the application, which is measured by the claims, nor is it intended to be limiting as to the scope of the present invention in any way.

### IV. BRIEF DESCRIPTION OF THE INVENTION

The present invention is an ingestible formulation for a plaque eliminator for animals that works systemically and comprises a formulation of a chlorophyll derivative combined with Spirulina and alfalfa wherein the chlorophyll derivative is at least 90 percent of the formulation.

Further, in the preferred embodiment the chlorophyll derivative is chlorophyllin. The chlorphyllin should be fresh when used in the inventive formula. The Inventor has discovered that using chlorophyll that is 180 days or less from harvest to use in a product increases the efficacy of the final product.

The inventive formulation has a dissolution time of greater than 60 minutes with a preferred dissolution time of 90 minutes in order to insure that the active elimination ingredients pass the stomach and can be absorbed in an effective state and is intended to be given to an animal on a daily basis to provide consistent and effective plaque elimination, with a steady state efficacy period of 48 to 72 hours.

The formulation is dose dependent and will vary depending on the size of the animal to which it is given.

The formulation may be a capsule, liquid, tablet, or gelcap when in its usable form with the preferred embodiment being in a capsule formulation. When produced in tablet form, the formulation may also include a tableting agent.

### V. BRIEF DESCRIPTION OF THE DRAWINGS

FIG. 1 is a chart ill'strating the effectiveness of the inventive formula against Streptococcus Sanguis.

FIG. 2 is a chart illustrating the ineffectiveness of the inventive formula against Streptococcus Salivarious.

FIG. 3 is a chart illustrating the ineffectiveness of the inventive formula against Prevotella Nigrescens.

### VI. DETAILED DESCRIPTION OF THE PREFERRED EMBODIMENT

Before explaining the preferred embodiment of the present invention in detail, it is to be understood that the present invention is not limited in its application to the details set forth in the following description. The present invention is capable of other embodiments and of being practiced and carried out in various ways. Also, it is to be understood that the phraseology and terminology employed herein are for the purpose of description and should not be regarded as limiting.

The present invention is a plaque eliminator designed for animals. The inventive formulation comprises a combination of a chlorophyll derivative, blue-green algae, and a balancing agent.

Chlorophyll is the green pigment found in higher plants, as well as algae. Chlorophyll is the principal photoreceptor in photosynthesis, the light-driven process in which carbon dioxide is “fixed” to yield carbohydrates and oxygen. Chlorophyll is a cyclic tetrapyrolle, similar in structure to the heme group of globins (hemoglobin, myoglobin) and cytochromes.

There are several types of chlorophyll. Higher plants and green algae, such as chlorella (see Chlorella) contain chlorophyll a and chlorophyll b in the approximate ratio of 3:1. Chlorophyll a is found with chlorophyll c in many types of marine algae. Red algae contain principally chlorophyll a and also chlorophyll d.

Research has shown that an effective chlorophyll derivative is chlorophyllin. Chlorophyllin is a semi-synthetic derivative of chlorophyll typically found sodium/copper, zinc/copper, or magnesium/copper forms. In contrast to chlorophyll, chlorophyllin is water-soluble. Chlorophyllin, like chlorophyll, has deodorizing activity.

The Inventor has discovered that any form of Chlorophyllin is suitable and that the use of fresh Chlorophyllin is best. Traditionally, products that do use a chlorophyll in them use processed chlorophyll. The Inventor has discovered that using chlorophyll that is 180 days or less from harvest to use in a product increases the efficacy of the final product. (Throughout the specification, the term chlorophyll, when used, is meant to also include a chlorophyll derivative and, especially, chlorophyllin, and the use of chlorophyll derivative or chlorophyllin is meant to include chlorophyll.)

Spirulina is an edible blue-green algae with a long history of safe human consumption. This tiny aquatic plant offers 60% all-vegetable protein, essential vitamins and phytonutrients such as the antioxidant beta carotene, the rare essential fatty acid GLA, sulfolipids, glycolipids and polysaccharides. Spirulina is rich in gamma-linolenic acid (GLA), and also provides alpha-linolenic acid (ALA), linoleic acid (LA), stearidonic acid (SDA), eicosapentaenoic acid (EPA), docosahexaenoic acid (DHA), and arachidonic acid (AA). Spirulina also contains vitamin B1 (thiamine), B2 (riboflavin), B3 (nicotinamide), B6 (pyridoxine), B9 (folic acid), B12 (cyanocobalamin), vitamin C, vitamin D, and vitamin E, and is a rich source of potassiun, calcium, chromium, copper, iron, magnesium, manganese, phosphorus, selenium, sodium, and zinc. Spirulina is also a source for chlorophyll-a, among other pigments.

Alfalfa is an excellent source of nutrients. Among its many benefits, it is known to act as a detoxifier and blood purifier and has been found to be beneficial for breath odor, liver disorders, infections, and disorders of the bones, joints and skin.

The inventor has discovered that while other available plaque eliminators may use chlorophyll or a derivative and/or Alfalfa, they do not generally work systemically, have ineffective ranges of ingredients, and use aged ingredients which have lost much if not all of their beneficial properties. Unlike the prior art, the present invention is formulated to work on a systemic level to eliminate breath and body odor.

A study conducted by the inventor on the inventive formula focused on bacterial species found in the oral cavity. Among the microorganisms studied were various streptococci, actinomyces, lactobacilli, and staphylococci. Also studied were black-pigmented bacteria that produce volatile sulfur compounds (VSC; hydrogen sulfide, methyl mercaptan and dimethyl sulfide) as a result of sulfur-containing amino acid (cysteine and cystine) metabolism and are the primary causes of plaque.

The bacteria were cultured in Mueller-Hinton (MH) broth in a 5% CO2 incubator at 37° C. for 24 h. 105 cells/ml were inoculated into 1 ml of MH broth and dispensed into 96 well microtiter plates (180 ul in each well). The inventive formula, studied in a tablet material form, was dissolved in deionized H₂O at the stated concentration and added to the wells (20 ul); the plates were then incubated for 18 h. The minimum inhibitory concentration (MIC) was determined by serial 2-fold dilutions of the product in the presence and in the absence of the material. MIC is defined as the concentration of the product that completely inhibits growth during the 18 h of incubation. Growth was assayed with a microplate reader (Molecular Devices, Menlo Park, Calif.) by following absorbance at 540 nm.

Control Specimens were placed in MH broth not containing antimicrobial powder (negative control); and CHX at 0.12% (as a positive control).

Results of the study showed the ability of the inventive formula to inhibit S. mutans which, in turn, is indicative of a strong ability to provide anti-caries activity. In addition, it is important to ascertain the ability of an antimicrobial agent to inhibit plaque formation particularly by early plaque colonizing organisms such as S. sanguis and A. viscosuis.

The study also confirmed that the inventive formula did not provide anti-bacterial effects against certain beneficial oral biofilm agents. Other important oral biofilm species examined in this study included Lactobacci caseii, S. salivarius and P. nigrescens. P. nigrescens was used in place of P. intermedius as it grew better and is also a strong VSC and odor-producing oral microbe. S. salivarius was used in place of S. aureus as it is a much more common oral bacterium. Inhibition of growth activity would reduce the ability of any of these microbes to adhere to salivary pellicle-coated tooth surfaces.

The chlorophyll extract (test product) was assessed for growth inhibition of 6 different oral bacteria using a spectrophotometric assay. As noted above, the assay consisted of incubation of cultures of S. mutans, S. sanguis, A. viscosus, L. caseii, S. salivarius or P. nigrescens with the extract for 24 hours and the measurement of growth using spectrophotometry at 540 nm. Controls consisted of bacteria with PBS or media only with the product. The following figure is representative of a typical microtiter plate used in this study.

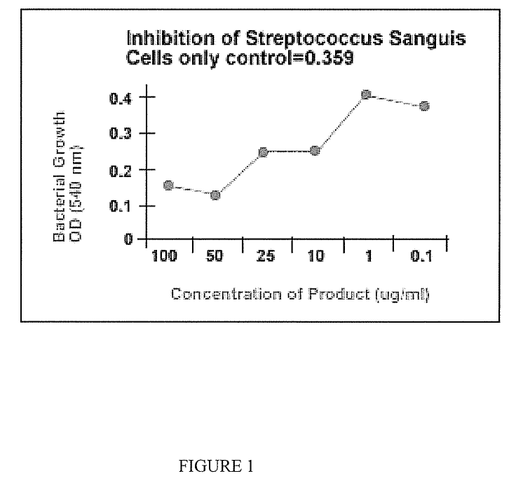

The results are shown in FIGS. 1, 2, and 3, each of which contains a graphical presentation of the results observed with that particular microorganism. Briefly, FIG. 1 illustrates the results of the study showing that the inventive formulation inhibited S. sanguis. The diluted extract (as low as 10 ug/ml) significantly inhibited the growth of S. sanguis.

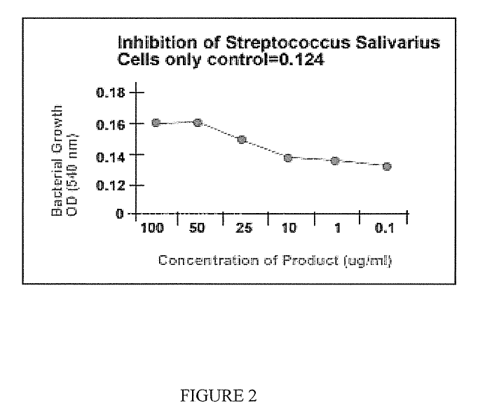

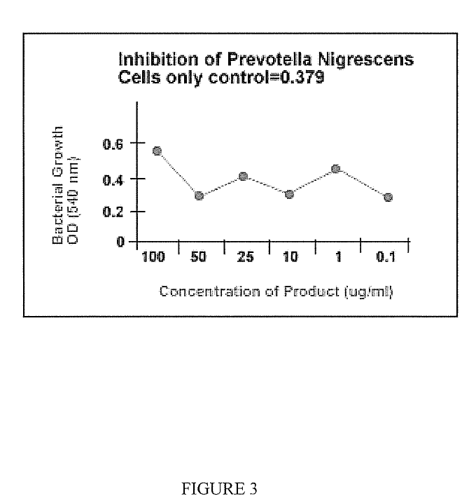

However, the extract did not inhibit Lactobacci caseii, S. salivarius, S. mutans and P. nigrescens at any concentration (See, e.g., FIGS. 2 and 3).

Central to the present invention is the amount of Chlorophyll or a derivative that is used. To be effective, the inventor has found that a level of at least 90% chlorophyll, chlorophyllin, or another chlorophyll derivative should be incorporated into a product. The balance of the formulation is made up of Spirulina and Alfalfa.

For example, a formulation that can be used in animals of less than 35 pounds may be comprised of:

| | |
|---|---|
| Organic Chlorophyllin | 65 mg |
| Organic Spirulina | 2 mg |
| Organic Alfalfa | 2 mg |

and a formulation that can be used in animals of more than 100 pounds may be comprised of:

| | |
|---|---|
| Organic Chlorophyllin | 130 mg |
| Organic Spirulina | 2 mg |
| Organic Alfalfa | 2 mg |

As illustrated above, a novel feature of the present invention is that the inventor has discovered that using dosing levels increases the efficacy of the product, an approach that the prior art has not taken.

When creating a tablet formula, there is need for a tableting agent which does not effect the formulation. The inventor has found that the following agents are suitable: dicalciim phosphate, microcrystalline cellulose, stearic acid, croscarmellose sodium, magnesium stearate, silicon dioxide, and agueous coating. Although the exact tableting agent is not critical and any agent that is ingestible and does not effect the formulation can be used.

The inventive formulation also provides significant advantage in that it has a dissolution time superior to that of the prior art. Prior art products generally have a dissolution time of 12 to 20 minutes. Such a short dissolution time does not provide adequate time for the product to work before breaking down. The inventive formula, on the other hand, has a dissolution time of such a length that the final product passes the stomach before breaking down. This helps to insure that effective amounts of ingredients having potency are biologically available for use. The inventive formula has a dissolution time ranging from about 60 minute to about 120 minutes with a preferred dissolution time of about 90 minutes. While a dissolution time range is provided herein, those skilled in the arts will immediately understand that the exact dissolution time is not critical to the invention. Instead, it is the fact that effective amounts of the formula's ingredients are biologically available passed the stomach where they can be appropriately absorbed in the gastrointestinal tract as effective ingredients.

The Inventor has also discovered that providing an animal the inventive formula on a consistent basis provides maximum and sustained effect with a steady state efficacy period of 48 to 72 hours.

The inventor's research has shown that the use of organic ingredients, although not critical to the invention, increases the efficacy of the finished product. More importantly, the inventor has discovered that the use of fresh ingredients dramatically improves the efficacy of the finished product.

It is to be understood, however, that even though numerous characteristics and advantages of the preferred and alternative embodiments have been set forth in the foregoing description, together with details of the structure and function of the embodiments, the disclosure is illustrative only.

Such variations should be considered to be within the scope and spirit of the invention which is meant to cover a plaque eliminating product for animals that is ingested comprising a formulation of at least 90% chlorophyll derivative. Changes may be made in detail within the principles of the invention to the full extent indicated by the broad general meaning of the terms in which the appended claims are expressed.

I claim:

1) A plaque eliminator for animals comprising an ingestible formulation further comprising a consumable blue-green algae, a balancing agent, and a chlorophyll selected from the group of chlorophyll and chlorophyll derivative.

2) The plaque eliminator for animals of claim wherein the chlorophyll derivative is chlorophyllin.

3) The plaque eliminator for animals of claim 1 wherein the chlorophyll is at least 90% of the formula composition.

4) The plaque eliminator for animals of claim 1 wherein the blue-green algae is Spinilina.

5) The plaque eliminator for animals of claim 1 wherein the balancing agent is alfalfa.

6) The plaque eliminator for animals of claim 1 wherein the chlorophyll, consumable blue-green algae, and balancing agent are organically grown.

7) The plaque eliminator for animals of claim 1 wherein the chlorophyll has a harvest-to-manufacture time period of 180 days or less.

8) The plaque eliminator for animals of claim 1 wherein the formulation is in a form selected from the group of capsule, tablet, liquid, and gelcap.

9) The formulation of claim 8 wherein the tablet formulation further includes a tableting agent.

10) The formulation of claim 9 wherein the tableting agent is selected from the group of dicalcium phosphate, microcrystalline cellulose, stearic acid, croscarmellose sodium, magnesium stearate, silicon dioxide, and agueous coating.

11) The plaque eliminator for animals of claim 1 wherein a dose for animals weighing less than 35 pounds includes about 65 mg of the chlorophyll, about 2 mg of Spirulina, and about 2 mg of alfalfa.

12) The plaque eliminator for animals of claim 1 wherein a dose for animals weighing more than 100 pounds includes about 130 mg of chlorophyll derivative, about 2 mg of Spirulina, and about 2 mg of alfalfa.

13) The plaque eliminator for animals of claim 1 having a dissolution time of a length such that the formulation does not begin to break down until the formulation has passed an animal's stomach.

14) The dissolution time of claim 13 having a range of about 60 minutes to about 120 minutes.

15) The dissolution time of claim 14 having a dissolution time of about 90 minutes.

16) A plaque eliminator for animals for systemic use comprising chlorophyllin in an amount greater than 90% of the total formulation when combined with Spirulina, and alfalfa.

17) The plaque eliminator for animals of claim 16 wherein the chlorophyllin has a harvest-to-manufacture time period of 180 days or less.

18) The plaque eliminator for animals of claim 16 wherein the formulation is in a form selected from the group of capsule, tablet, liquid, and gelcap.

19) The formulation of claim 18 wherein the tablet formulation further includes a tableting agent.

20) The tableting formulation of claim 19 wherein the tableting agent is selected from the group of dicalcium phosphate, microcrystalline cellulose, stearic acid, croscarmellose sodium, magnesium stearate, silicon dioxide, and agueous coating.

21) The plaque eliminator for animals of claim 16 wherein a dose for animals weighing less than 35 pounds includes about 65 mg of chlorophyll derivative, about 2 mg of Spirulina, and about 2 mg of alfalfa.

22) The plaque eliminator for animals of claim 16 wherein a dose for animals weighing more than 100 pounds includes about 130 mg of chlorophyll derivative, about 2 mg of Spirulina, and about 2 mg of alfalfa.

23) The plaque eliminator for animals of claim 16 having a dissolution time of a length such that the formulation does not begin to break down until the formulation has passed an animal's stomach.

24) The dissolution time of claim 23 having a range of about 60 minutes to about 120 minutes.

25) The dissolution time of claim 24 having a dissolution time of about 90 minutes.

26) A method for producing a plaque eliminator for animals comprising the steps of:
harvesting chlorophyll no more than 180 days before using it in the eliminator;
combining the chlorophyll with Spirulina and alfalfa wherein the amount of chlorophyll is not less than 90% of a finished formulation when compared to the Spirulina and alfalfa; and,
producing a finished product in a liquid, capsule, tablet, or gelcap form, which finished product form depends on the end use for the finished product.

27) The method of claim 26 including the further step of substituting a chlorophyll derivative made from chlorophyll that has been harvested no more than 180 days from use.

28) The method of claim 27 wherein the chlorophyll derivative is chlorophyllin.

29) The method of claim 26 including the further step of producing a finished product having a dissolution time of a length sufficient to insure the product does not break down before the product passed an animal's stomach.

30) The method of claim 29 wherein the dissolution time is in the range of about 60 minutes to about 120 minutes.

31) The method of claim 30 wherein the dissolution time is 90 minutes.
